(19) United States
(12) Patent Application Publication
(10) Pub. No.: US 2005/0118228 A1
(43) Pub. Date: Jun. 2, 2005

(54) Compositions and methods for augmentation or repair of intervertebral discs

(76) Inventor: Hai H. Trieu, Cordova, TN (US)

Correspondence Address: WOODARD, EMHARDT, MORIARTY, MCNETT & HENRY LLP;BANK ONE TOWER/CENTER, 111 MONUMENT CIRCLE, SUITE 3700, INDIANAPOLIS, IN, 46204-5137, US

(21) Appl. No.: 11/030,706

(22) Filed: Jan. 6, 2005

Related U.S. Application Data
(63) Continuation-in-part of application No. 10/402,723, filed on Mar. 28, 2003.

(51) Int. Cl. A61K 45/00; A61F 2/44; C12N 5/08

(52) U.S. Cl. 424/423; 424/93.7; 623/17.11; 435/368

(57) ABSTRACT

A method of augmenting and/or repairing an intervertebral disc by administering stem cell material into the disc. The stem cells may be undifferentiated cells, or they may be cells that have differentiated and have subsequently been dedifferentiated. The stem cells may be induced to express at least one characteristic of human intervertebral disc cells, such as fibroblast cells, chondrocyte cells, or notochordal cells, by exposing them to agents and/or environments calculated to induce the desired differentiation. In some embodiments, the stem cell material may be provided in conjunction with a collagen-based material, which may be a collagen-rich lattice or particles of collagen material. The stem cell material may be provided as a stem cell isolate, which may be substantially free of non-stem cell material. Other therapeutic agents may be administered with the stem cell material.

### REFERENCE TO RELATED APPLICATIONS

This application is a continuation-in-part and claims priority from U.S. patent application Ser. No. 10/402,723, filed Mar. 28, 2003, which is incorporated herein by reference in its entirety.

### FIELD OF THE INVENTION

The present invention relates generally to materials and methods for augmenting and/or repairing intervertebral discs, and more particularly to materials and methods for augmenting and/or repairing intervertebral discs with stem cell material.

### BACKGROUND OF THE INVENTION

A healthy intervertebral disc facilitates motion between pairs of vertebrae while absorbing and distributing shocks. The disc is composed of two parts: a soft central core (the nucleus pulposus) that bears the majority of the load, and a tough outer ring (the annulus fibrosis) that holds and stabilizes the core material.

As the natural aging process progresses, the disc may dehydrate and degenerate, adversely affecting its ability to adequately cushion and support the vertebral bodies. This natural desiccation, which in its more advanced state is often referred to as “black disc” because of the disc's dehydrated appearance on Magnetic Resonance Imaging [MRI], can cause discomfort to the patient as the vertebrae to come closer together—compressing the spinal nerves and causing pain.

Techniques for addressing degenerative disc disease have heretofore relied primarily on disc replacement methods. In cases in which a dehydrated and/or degenerating disc was augmented before disc replacement was required, the augmentation materials have primarily been synthetic devices that expand, are inflated, or deploy expanding elements when implanted into the disc.

In recent studies, pluripotent and/or multipotent stem cells have been suggested as being potentially useful for medical applications. Pluripotent stem cells are self-renewing cells which are capable of differentiating into any one of more than 200 different cell types found in the body. Embryonic pluripotent and/or multipotent stem cells may be characterized as either embryonal carcinoma (“EC”) cells, embryonic germ (“EG”) cells, or embryonic stem (“ES”) cells. Non-embryonic pluripotent and/or multipotent stem cells may be obtained from adult somatic cell sources. Non-embryonic multipotent stem cells include, for example, neural stem cells, mesenchymal stem cells, bone marrow stem cells, and stem cells obtained from liposuction. For the purposes of this disclosure, embryonic pluripotent or multipotent cells, and non-embryonic pluripotent or multipotent cells, are all referred to as “stem cells.” In other words, any cell that has not differentiated into a mature cell type, may be referred to as a “stem cell” for the purposes of this disclosure.

Mesenchymal stem cells are adult multipotent cells derived from multiple sources, including bone marrow stroma, blood, dermis, and periosteum. These cells can be cultured continuously in vitro without spontaneous differentiation. However, under the proper conditions, mesenchymal stem cells can be induced to differentiate into cells of the mesenchymal lineage, including adipocytes, chondrocytes, osteocytes, tenocytes, ligamentogenic cells, myogenic cells, bone marrow stroma cells, and dermogenic cells.

Hematopoietic stem cells are multipotent cells capable of self renewal and differentiation into multiple blood cells types, including erythrocytes, megakaryocytes, monocytes/macrophages, granulocytes, mast cells, B-cells and T-cells. Hematopoietic stem cells can be obtained from fetal liver, adult bone marrow, or mononuclear muscle precursor cells called satellite cells.

Most recently, human pluripotent stem cells have been derived via the reprogramming of somatic cell nuclei via nuclear transfer to oocytes. That technique, called therapeutic cloning, allows pluripotent stem cells derived from the patient to be used in autologous transplant therapy. Similarly, multipotent stem cells can be created by dedifferentiation of adult (non-embryonic, non-fetal) mammalian tissues. However, to date, such multipotent or pluropotent cells have never been used to augment or repair an intervertebral disc.

Among the literature references relating to the use of stem cells to grow or treat tissue are U.S. patent Publications Nos. 2002/0076400, 2003/0054331, and 2004/0171146, all of which provide background to the present invention.

A need therefore remains for techniques for using pluripotent and/or multipotent stem cells to augment or repair an intervertebral disc. The present invention addresses that need.

### SUMMARY OF THE INVENTION

Briefly describing one aspect of the present invention, there is provided a method of augmenting and/or repairing an intervertebral disc by administering stem cell material into the disc. The stem cell material may be from undifferentiated cells, or it may be from cells that have differentiated and have subsequently been returned to their undifferentiated state. Regardless of whether the cells intended for implantation have begun to differentiate before selection for use in a disc space, in some embodiments the stem cell material comprises cells that have been induced to express at least one characteristic of human intervertebral disc cells (such as fibroblast cells, chondrocyte cells, or notochordal cells) before the material is implanted in a disc. Alternatively, undifferentiated stem cell material and a material capable of inducing stem cell differentiation may be combined just prior to, during, or after implantation in a disc space so that the stem cell material differentiates in the disc space to express at least one characteristic of human intervertebral disc cells.

In some embodiments, the stem cell material is provided in conjunction with a collagen-based material, which may be a collagen-rich lattice. The collagen-based material may be provided in dehydrated form, and rehydrated after administration, or it may be provided in a hydrated form, such as a slurry or gel. Cross-linking agents such as glutaraldehyde may be included in the collagen-based material to promote collagen crosslinking.

In addition, radio-contrast materials may be included to enhance imaging. Performance-enhancing additives such as analgesics and/or antibiotics may be included to provide additional therapeutic benefits.

In some preferred embodiments the stem cell material is provided as a stem cell isolate, which may be substantially free of non-stem cell material.

Objects and advantages of the claimed invention will be apparent from the following description.

### DESCRIPTION OF THE PREFERRED EMBODIMENTS

For the purposes of promoting an understanding of the principles of the invention, reference will now be made to certain preferred embodiments and specific language will be used to describe the same. It will nevertheless be understood that no limitation of the scope of the invention is thereby intended, such alterations and further modifications in the preferred embodiments being contemplated as would normally occur to one skilled in the art to which the invention relates.

As indicated above, one aspect of the present invention relates to materials and methods for using stem cell material to augment or repair an intervertebral disc. In the most preferred embodiments the stem cell material is administered to a disc nucleus that is contained in a substantially sound annulus. In other embodiments the material is administered to a disc nucleus that is contained in a damaged or defective annulus.

In one aspect of the present invention a stem cell material is administered to an intervertebral disc and is induced to differentiate into an intervertebral disc cell. The stem cell material may comprise stem cells that have never differentiated, such as embroyonic stem cells, or it may comprise stem cells that have differentiated and have subsequently been dedifferentiated to restore their multipotent or pluripotent capacity.

Regardless of which type of stem cell is used, the cells may be induced to express one or more characteristics of human intervertebral disc cells. In one embodiment the cells are induced to exhibit characteristics of intervertebral disc cells before they are administered to the disc (for example, in vitro), while in other embodiments the cells are induced to exhibit intervertebral disc cell characteristics after they have been administered to the disc (in vivo).

In one preferred embodiment the stem cell material is a lipo-derived stem cell material. More preferably, the stem cell material is substantially free of other cell types (e.g., adipocytes, red blood cells, other stromal cells, etc.) and extracellular matrix material. Most preferably, the stem cell material is completely free of such other cell types and matrix material. The lipo-derived stem cell material may be derived from the adipose tissue of a primate, and is most preferably derived from the person receiving the spinal implant. While the stem cell material can be of any type of stem cell, desirably the material is of mesodermal origin.

When adipose-derived stem cell material is used, the material can be obtained by any suitable method. In general though, the procedure begins by isolating adipose tissue from the source animal. When human adipose stromal cells from living donors are used, well-recognized protocols such as surgical or suction lipectomy are preferred.

After obtaining the adipose tissue, it is preferably processed to separate stem cells from the remainder of the material. In one preferred embodiment the adipose tissue is washed with a physiologically-compatible saline solution (e.g., phosphate buffered saline), and is then vigorously agitated and left to settle. That procedure removes loose matter (e.g., damaged tissue, blood, erythrocytes, etc.) from the adipose tissue. The washing and settling steps may be repeated until the supernatant is relatively clear of debris.

The remaining cells generally will be present in lumps of various size, so the material is preferably processed to degrade the gross structure while minimizing damage to the cells themselves. One method of achieving this is to treat the washed lumps of cells with an enzyme that weakens or destroys bonds between cells (e.g., collagenase, dispase, trypsin, etc.). The amount and duration of such enzymatic treatment will vary, depending on the conditions employed, but the use of such enzymes is generally known in the art. Alternatively or in conjunction with enzymatic treatment, the lumps of cells can be degraded using other treatments, such as mechanical agitation, sonic energy, thermal energy, etc.

The degradation step typically produces a slurry or suspension of aggregated cells (generally liposomes) and a fluid fraction containing generally free stromal cells (e.g., red blood cells, smooth muscle cells, endothelial cells, fibroblast cells, and stem cells). The next step in the separation process is therefore to separate the aggregated cells from the stromal cells. This can be accomplished by centrifugation, which forces the stromal cells into a pellet covered by a supernatant. The supernatant then can be discarded and the pellet suspended in a physiologically-compatible fluid.

The suspended cells typically include erythrocytes, and in most protocols it is desirable to lyse these. Methods for selectively lysing erythrocytes are known in the art, and any suitable protocol can be employed (e.g., incubation in a hyper- or hypotonic medium). If the erythrocytes are lysed, the remaining cells should then be separated from the lysate, typically by filtration or centrifugation. Regardless of whether the erythrocytes are lysed, the suspended cells can be washed, re-centrifuged, and resuspended one or more successive times to achieve greater purity.

Adult stem cells can be separated using a cell sorter or on the basis of cell size and granularity, stem cells being relatively small and agranular. They can also be separated immunohistochemically, for example, by panning or using magnetic beads. Any of the steps and procedures for isolating the inventive cells can be performed manually, if desired. Alternatively, the process of isolating such cells can be facilitated through a suitable device, many of which are known in the art.

As indicated above, in some embodiments the stem cell material comprises cells that have differentiated and subsequently been dedifferentiated to restore their multipotent capacity. One method for accomplishing that comprises establishing a culture of the cells and treating the cells to reverse specific epigenetic chromosomal changes associated with differentiation.

Heritable changes in gene expression that occur during cell differentiation are due in part to epigenetic changes in chromosomal conformation. Further, loosely condensed regions of chromosomes contain transcriptionally active genes and highly condensed regions of chromosomes contain transcriptionally silenced genes. The state of chromosome condensation and transcription activity is controlled in part by DNA methylation and histone acetylation. Methylation or hypermethylation of cytosines within CpG promoters is associated with gene silencing, whereas unmethylated DNA is generally transcriptionally active.

Differentiated adult somatic cells show stable and specific patterns of methylation, whereas pluripotent cells, such as primordial germ cells and preimplantation embryos, show genome-wide patterns of demethylation. A few studies have demonstrated that these heritable patterns of methylation can be reversed. For example, murine thymic lymphocytes have been fused with murine embryonic germ cells and demonstrated the genome-wide demethylation of the lymphocyte cell nucleus. The resulting demethylated nucleus was subsequently shown to be pluripotent.

Accordingly, in one aspect of the present invention adult somatic cells are reprogrammed using DNA demethylation. Demethylation provides the inhibition of methylation of nucleotides comprising DNA. According to the present invention, adult somatic cells may be treated with an agent to promote or induce the demethylation of DNA. In one embodiment of the demethylation step, adult somatic cells are treated with 5-aza-2′-deoxycytidine. Primary adult somatic cells are cultured in normal growth medium in the presence of 0.1 to 100 .mu.M of 5-aza-2′-deoxycytidine (Sigma Chemical Co., St. Louis, Mo.), for 1 to 10 days, preferably 5 days, to promote or induce demethylation of DNA. Other reagents may be used in the demethylation step, including, for example, methylase specific antibodies or other inhibitors of methylases.

In addition to specific patterns of DNA methylation and demethylation, global patterns of transcription are also regulated by chromatin remodeling enzymes, such as histone acetylases and deacetylases. Acetylated histones bind to DNA with lower affinity than deacetylated histones, thereby generally permitting transcription factors to bind to DNA. Conversely, deacetylated histones bind DNA with higher affinity, blocking the access of transcription activators to DNA, thereby generally repressing transcription. In another embodiment of the invention, primary adult somatic cells are reprogrammed via inhibition of or reversal of histone deacetylation. Primary adult somatic cells are cultured in normal growth medium in the presence of 0.1-10,000 ng/ml of trichostatin A (Sigma Chemical Co., St. Louis, Mo.) for at least 24 hours. Trichostatin A treatment of cells has been shown to induce or allow the expression of previously silenced genes. Alternatively, cells may be treated with sodium butyrate, which also inhibits histone deacetylation. Any reagent which induces or facilitates changes in histone acetylation or DNA methylation may be used to accomplish that.

In yet another embodiment, primary adult somatic cells are treated with a chromatin remodeling protein preferably nucleoplasmin, which is a nuclear chaperone that facilitates the exchange of histone H1 with histone B4 and HMG1, thereby facilitating activation of transcription. In one preferred embodiment, a transit peptide (e.g., Tat) is fused to a peptide comprising nucleoplasmin which is administered to cells in normal medium. Histone exchange is allowed to proceed before the nucleoplasmin treatment is stopped. It is envisioned that cells may be treated with any chromatin remodeling enzyme, reagent, intercalating agent, or combination thereof, that is known in the art, which facilitates the removal of transcription repressors and nuclear remodeling.

In another embodiment, primary adult somatic cells are treated with a combination of demethylation agents, deacetylation inhibitors or acetylation promoters and/or nuclear chaperones to promote nuclear reprogramming. As used herein, the term “nuclear chaperone” means any reagent that facilitates the exchange of histone H1 or other transcription repressors for HMG1, histone B4 or other transcription activators. It is envisioned that any reagent which induces or facilitates changes in histone acetylation or DNA methylation may be used in the practice of this invention. It is also envisioned that cells may be treated with any chromatin remodeling enzyme, reagent, intercalating agent, or combination thereof, that is known in the art, which facilitates the removal of transcription repressors and nuclear remodeling.

Additionally, the skilled artisan may treat the primary cells with other reagents known in the art to block DNA methylation, promote DNA demethylation, block histone deacetylation, promote histone acetylation, and/or promote the exchange of histone H1 with histone B4 or HMG1, in order to reprogram the genome of said cells.

Cultures of adult somatic stem cells may be treated with either one or more of the following reagents to induce metaphase arrest: G2-M cyclins, for example cyclin-A or cyclin-B, c-Mos, colchicine, colcemid or any other reversible microtubule drug. Polypeptide reagents, such as cyclin-A, cyclin-B or c-Mos, may be administered to cells through membrane translocation methods including, but not limited to, microinjection, liposome-mediated translocation, or direct translocation of polypeptides which are fused to transit peptides. Alternatively, vectors comprised of polynucleotides encoding cyclin-A, cyclin-B or c-Mos, for example, under the control of a regulated promoter, such as the commercially available Tet-on/Tet-off system (Clontech, Palo Alto Calif.), may be transfected into cultured cells via cationic lipid transduction, microinjection, or electroporation. After metaphase arrest is sustained in the cell for at least 1 to 6 hours, the cell may be released from metaphase arrest by media replacement, as in the case of treatment by peptide or microtubule poison, or by promoter repression, as in the case of polynucleotide vector transfection.

It is to be appreciated that easily obtainable adult somatic cells, most preferably hair outer root sheath (ORS) cells, epidermal keratinocytes or buccal epithelial cells may be obtained from a subject and expanded in culture, as described herein, wherein the subject is preferably a human. The cells are treated with an amount of a demethylation agent, preferably about 10 .mu.M 5-aza-2′-deoxycytidine for about 5 days, to induce global genomic demethylation. These cells may also be treated with a deacetylation inhibitor or acetylation promoter, preferably 100 ng/ml or 1 .mu.M of trichostatin A for about 24 hours, to promote histone acetylation. These cells may also be treated with an amount of a polypeptide comprising a nuclear chaperone or other chromatin remodeling enzyme (Fry and Peterson, supra), preferably nucleoplasmin or tat-nucleoplasmin, to facilitate the removal of transcription repressors from the DNA.

Subsequent to the aforementioned step or steps, the cells are then treated with an amount of an agent that arrests cells in metaphase, preferably a polypeptide comprising cyclin-A or cyclin-B, for 30 hours to induce prolonged mitotic arrest. The cells are then released from the mitotic arrest by washing the cells in at least one change of culture medium.

Subsequent to the mitotic arrest step, adherent cells are trypsinized, replated and cultured in media designed to support growth of stem cells. In a preferred embodiment, the remodeled cells are passaged onto a layer of mouse embryo fibroblast feeder cells in 80% KNOCKOUT.RTM. DMEM, 20% KNOCKOUT.RTM. SR (GIBCO/BRL, Bethesda Md.), 1 mM glutamine, 0.1 mM .beta.-mercaptoethanol, 1% nonessential amino acid stock (GIBCO/BRL, Bethesda Md.), 4 ng/ml basic fibroblast growth factor, and 1,000 U/ml leukemia inhibitory factor (ES cell medium). The KNOCKOUT.RTM. DMEM and KNOCKOUT.RTM. SR are special formulations designed to enhance the growth and maintain the pluripotentiality of embryonic stem cells. The skilled artisan may also use other cell media formulations, which are known in the art, to propagate pluripotent cells.

After the stem cells are provided, in one aspect of the present invention the cells are induced to express one or more characteristics of human intervertebral disc cells. That process can occur in vitro or in vivo, as will be discussed more fully below. As previously mentioned, these cells that have begun to express characteristics of mature human intervertebral disc cells are still referred to as “stem cells.” In fact, any cell that has not differentiated into a mature cell type, may be referred to as a “stem cell” for the purposes of this disclosure.

In one preferred embodiment, remodeled cells are directly cultured under conditions that are not optimal for maintaining stem cells, but rather allow the remodeled cells to differentiate. Generally, such culture conditions may lack serum, lack feeder cells, contain a high density of cells, or contain one or more of various morphogenic growth or differentiation factors, such as media used to culture mature cells of a defined phenotype, mature cells of a desired and/or defined phenotype, or specific differentiation factors such as, for example, retinoic acid or nerve growth factor.

One type of treatment is to culture the inventive cells in culture media that has been conditioned by exposure to mature cells (pr precursors thereof) of the respective type to be differentiated (e.g., media conditioned by exposure to myocytes can induce myogenic differentiation, media conditioned by exposure to heart valve cells can induce differentiation into heart valve tissue, etc.).

Expanded cultures of in vitro derived adult pluripotent stem cells may be differentiated by in vitro treatment with growth factors and/or morphogens. Of course, defined media for inducing differentiation also can be employed. Chondrogenic differentiation can be induced by exposing the cells to between about 1 μM to about 10 μM insulin and between about 1 μM to about 10 .mu.M transferrin, between about 1 ng/ml and 10 ng/ml transforming growth factor (TGF) beta. 1, and between about 10 nM and about 50 nM ascorbate-2-phosphate (50 nM). For chondrogenic differentiation, preferably the cells are cultured in high density (e.g., at about several million cells/ml or using micromass culture techniques), and also in the presence of low amounts of serum (e.g., from about 1% to about 5%). Osteogenic developmental phenotype may be induced by exposing the cells to between about 10⁻⁷ M and about 10⁻⁹ M dexamethasone (e.g., about 1 .mu.M) in combination with about 10 .mu.M to about 50 .mu.M ascorbate-2-phosphate and between about 10 nM and about 50 nM .beta.-glycerophosphate, and the medium also can include serum (e.g., bovine serum, horse serum, etc.).

After culturing the cells in the differentiating-inducing medium for a suitable time (e.g., several days to a week or more), the cells can be assayed to determine whether, in fact, they have differentiated to acquire physical qualities of a given type of cell. One measurement of differentiation per se is telomere length, undifferentiated stem cells having longer telomeres than differentiated cells; thus the cells can be assayed for the level of telomerase activity. Alternatively, RNA or proteins can be extracted from the cells and assayed (via Northern hybridization, rtPCR, Western blot analysis, etc.) for the presence of markers indicative of the desired phenotype. Of course, the cells can be assayed immunohistochemically or stained, using tissue-specific stains. Similarly, ostogenesis can be assessed by staining the cells with bone-specific stains (e.g., alkaline phosphatase, von Kossa, etc.) or probed for the presence of bone-specific markers (e.g., osteocalcin, osteonectin, osteopontin, type I collagen, bone morphogenic proteins, cbfa, etc.). Chondrogenesis can be determined by staining the cells using cartallge-specific stains (e.g., alcian blue) or probing the cells for the expression/production of cartilage-specific molecules (e.g., sulfated glycosaminoglycans and proteoglycans (e.g., keratin, chondroitin, etc.) in the medium, type II collagen, etc.). Other methods of assessing developmental phenotype are known in the art, and any of them is appropriate. For example, the cells can be sorted by size and granularity. Also, the cells can be used to generate monoclonal antibodies, which can then be employed to assess whether they preferentially bind to a given cell type. Correlation of antigenicity can confirm that the stem cell has differentiated along a given developmental pathway.

Alternatively, stem cells may be induced in vivo to express one or more characteristics of human intervertebral disc cells. That can be accomplished by several methods, including providing exogenous stem cells in the intervertebral disc, preferably in high density. Stem cells may be added either with or without agents selected to induce development to human intervertebral disc cells.

Among the factors that may induce stem cells to differentiate into human intervertebral disc cells are factors used to differentiate stem cells in vitro. For example, transforming growth factor (TGF)-β, ascorbate-2-phosphate, bone morphogenetic proteins, fibroblast growth factors, platelet-derived growth factors, β-glycerophosphate, insulin, insulin-like growth factors, transferrin, hydrocortisone, and others that may be known by persons skilled in the art, may be used for that purpose.

Regardless of whether the cells are provided with one or more of the inducing agents suggested above, it is understood that the cells may be cultured or grown in an environment that has been conditioned by exposure to mature cells (or precursors thereof) of the respective type to be differentiated (e.g., media conditioned by exposure to myocytes can induce myogenic differentiation, media conditioned by exposure to heart valve cells can induce differentiation into heart valve tissue, etc.). Such treatment is particularly effective when the cells are provided in high density.

In other aspects of the present invention, to facilitate the use of the stem cell material to augment a spinal disc the stem cell material is provided in a biologically compatible lattice material. Typically, the lattice comprises collagen-rich material from the same adipose tissue as provided the stem cell material. Desirably, the lattice is biodegradable over time, so that it will be absorbed into the body as the stem cell material it develops. The lattice can also include hormones, such as growth factors, cytokines, and morphogens (e.g., retinoic acid, aracadonic acid, etc.), desired extracellular matrix molecules (e.g., fibronectin, laminin, collagen, etc.), or other materials (e.g., DNA, viruses, other cell types, etc.) as desired.

To form the stem cell/collagen-rich lattice material, stem cells are introduced into the lattice such that they permeate into the interstitial spaces therein. For example, the lattice can be soaked in a solution or suspension containing the cells, or they can be infused or injected into the lattice. A particularly preferred composition is a hydrogel formed by crosslinking of a suspension including the collagen-rich lattice material with the stem cell material dispersed therein. This method of formation permits the cells to be dispersed throughout the lattice, facilitating more even permeation of the lattice with the cells.

Lattices suitable for inclusion into the composition can be derived from any suitable source (e.g., matrigel), and some commercial sources for suitable lattices exist (e.g., suitable polyglycolic acid can be obtained from sources such as Purac Biochem., and Boehringer Ingelheim). As indicate above, the preferred source of the collagen-rich lattice is the acellular portion of the adipose tissue that provided the stem cells—i.e., adipose tissue extracellular matrix matter substantially devoid of cells. Typically, such lipo-derived lattice includes proteins such as proteoglycans, glycoproteins, hyaluronins, fibronectins, collagens (type I, type II, type III, type IV, type V, type VI, etc.), and the like, which serve as excellent substrates for cell growth. Additionally, the lipo-derived lattices can include hormones, preferably cytokines and growth factors, for facilitating the growth of cells seeded into the lattice.

The lipo-derived lattice can be isolated form adipose tissue similarly as described above, except that it will be present in the acellular fraction. For example, adipose tissue or derivatives thereof (e.g., a fraction of the cells following the centrifugation as discussed above) can be subjected to sonic or thermal energy and/or enzymatic processed to recover the lattice material. Also, desirably the cellular fraction of the adipose tissue is disrupted, for example by treating it with lipases, detergents, proteases, and/or by mechanical or sonic disruption (e.g., using a homogenizer or sonicator). However isolated, the material is initially identified as a viscous material, but it can be subsequently treated, as desired, depending on the desired end use. For example, the raw lattice material can be treated (e.g., dialyzed or treated with proteases or acids, etc.) to produce a desirable lattice material. Thus the lattice can be prepared in a hyrated form or it can be dried or lyophilized into a substantially anhydrous form or a powder. Thereafter, the powder can be rehydrated for use as a cell culture substrate, for example by suspending it in a suitable cell culture medium. In this regard, the lipo-derived lattice can be mixed with other suitable lattice materials, such as described above.

In some embodiments the stem cells or the stem cell/collagen-rich lattice material are combined with additional collagen-based material for use to augment a spinal disc. The additional collagen-based material is preferably derived from natural, collagen-rich tissue, such as intervertebral disc, fascia, ligament, tendon, demineralized bone matrix, etc. The material may be autogenic, allogenic, or xenogenic, or it may be of human-recombinant origin. In alternative embodiments the collagen-based material may be a synthetic, collagen-based material. Examples of preferred collagen-rich tissues include disc annulus, fascia lata, planar fascia, anterior or posterior cruciate ligaments, patella tendon, hamstring tendons, quadriceps tendons, Achilles tendons, skins, and other connective tissues.

The stem cell/collagen-rich lattice material, with or without additional collagen-based material, may be provided in any form appropriate for introduction into a disc space. For example, the material may be a solid, porous, woven, or non-woven material. The material may be provided as particles or small pieces, or as a fibrous material.

In some embodiments the collagen-based material provided with the stem cells is provided in a dehydrated state, and is “rehydrated” after implantation in the disc. In other embodiments the collagen-based material is implanted “wet.” When the material is “wet,” it may be that way because it has never been dehydrated, or it may have been dehydrated and reconstituted. When reconstituted, the material may be reconstituted with saline or another aqueous medium, or it may be reconstituted with a non-aqueous medium such as ethylene glycol or another alcohol. Moreover, when provided in a “wet” state, the material may be provided as a gel, solution, suspension, dispersion, emulsion, paste, etc. In the most preferred embodiments the material is a particulate and/or fibrous material suitable for injection through a hypodermic needle into a disc.

In the most preferred embodiments the collagen-rich lattice material and/or the additional collagen-based material are provided as particles ranging between 0.05 mm and 5 mm in size. When materials such as fascia lata or disc annulus particles are used as the additional collagen-based material the particles preferably range in size from 0.1 mm to 5 mm. When materials such as demineralized bone matrix are used the particles preferably range in size from 0.05 mm to 3 mm. When small plugs of material are used the plugs preferably range in size from 0.5 mm to 5 mm. In some embodiments larger sized pieces, such as pieces up to 20 mm in size, may be used.

The materials may be processed or fabricated using more than one type of tissue. For example, mixtures of stem cell/collagen-rich lattice material with fascia lata and/or demineralized bone matrix (DBM) may be preferred in appropriate cases, as may mixtures of stem cell/collagen-rich lattice material with DBM and annulus fibrosis material.

Cross-linking agents may be added to the formulation to promote cross-linking of the collagen materials. For example, glutaraldehyde or other protein cross-linking agents may be included in the formulation. The cross-linking agents may promote covalent or non-covalent crosslinks between collagen molecules. Similarly, agents to inhibit protein denaturization may also be included. Crosslinking agents that would be appropriate for use in the claimed invention are known to persons skilled in the art, and may be selected without undue experimentation.

When the material is to be used as a slurry or gel, additives to promote slurry or gel formation may also be included. These additives may promote protein folding, water binding, protein-protein interactions, and water immobilization.

In addition, a radiocontrast media, such as barium sulfate, or a radiocontrast dye, such as HYPAQUE®, may be included to aid the surgeon in tracking the movement and/or location of the injected material. Radiocontrast materials appropriate for use in discography are known to persons skilled in the art, and may be selected for use in the present invention without undue experimentation.

Finally, other additives to provide benefits to the injected stem cell/collagen-based material may also be included. Such additives include anesthetics, to reduce pain caused by the procedure, and antibiotics, to minimize the potential for bacterial infection.

Proteoglycans and/or other polysaccharides may also be included to attract and/or bind water to keep the nucleus hydrated. Similarly, growth factors and/or other cells (e.g., intervertebral disc cells, stem cells, etc.) to promote healing, repair, regeneration and/or restoration of the disc, and/or to facilitate proper disc function, may also be included. Additives appropriate for use in the claimed invention are known to persons skilled in the art, and may be selected without undue experimentation.

It is to be appreciated that the stem cell/collagen material may be processed into various forms (e.g. solid, porous, woven, non-woven, particulate, gel, solution suspension, paste, etc.) before being added to the disc space. In one preferred embodiment the materials is dehydrated before injection into the disc space, where it is rehydrated by absorbing fluid from the disc space. In other embodiments the collagen material is provided as a gel, slurry, or other hydrated formulation before implantation.

The stem cell material/collagen-based material is “surgically added” to the disc space. That is, the material is added by the intervention of medical personnel, as distinguished from being “added” by the body's natural growth or regeneration processes. The surgical procedure preferably includes injection through a hypodermic needle, although other surgical methods of introducing the collagen-based material into the disc may be used. For example, the material may be introduced into a disc by extrusion through a dilated annular opening, infusion through a catheter, insertion through an opening created by trauma or surgical incision, or by other means of invasive or minimally invasive deposition of the materials into the disc space.

As to the benefits of the inventive materials and methods, augmentation of the intervertebral disc may restore or improve the natural condition and/or performance of the disc. In addition, augmentation may retard or reverse the progressive degeneration of a dehydrated disc.

Reference will now be made to specific examples using the processes described above. It is to be understood that the examples are provided to more completely describe preferred embodiments, and that no limitation to the scope of the invention is intended thereby.

### EXAMPLE 1

### Obtaining Stem Cell Material From Somatic Tissue

Raw liposuction aspirate may be obtained from patients undergoing elective surgery. Prior to the liposuction procedures, the patient may be given epinephrine to minimize contamination of the aspirate with blood. The aspirate is then strained to separate associated adipose tissue pieces from associated liquid waste. Isolated tissue is rinsed thoroughly with neutral phosphate buffered saline and then enzymatically dissociated with 0.075% w/v collagenase at 37.degree. C. for about 20 minutes under intermittent agitation.

Following digestion, the collagenase is neutralized and the slurry is centrifuged at about 260 g for about 10 minutes. This produces a multi-layered supernatant and a cellular pellet. The supernatant is then removed and may be retained for further use. The pellet is resuspended in an erythrocyte-lysing solution and incubated without agitation at about 25.degree. C. for about 10 minutes. Following incubation, the medium is neutralized and the cells are again centrifuged at about 250 g for about 10 minutes.

Following the second centrifugation, cells in the pellet are suspended and assessed for viability by tryan blue exclusion and cell number. Cells are then plated at 1×10⁶ cells/100 mm dish and grown at 37 degree. C., 5% CO₂ in media supplemented with about 10% fetal bovine serum. The majority of the cells are adherent, small, mononucleic, fibroblast-like cells containing no visible lipid droplets. The majority of the cells stained negatively with oil-red 0 and von Kossa.

Cells may be assayed for the expression of telomerase (using a commercially available TRAP assay kit), along side HeLa and NH-12 cells included in the assay as positive controls. As a negative control telomerase activity is assayed in human foreskin fibroblasts and heated HN-12 cell extracts. Telomerase activity is measured by phosphoimaging telomeric products resolved using 12.5% polyacrylamide cells. Telometric ladders, indicative of telomerase activity, and consistent with the presence of stem cells were observed in the cells derived from adipose tissue and in the positive controls but not in the negative controls. These results confirm that stem cells may be isolated from adipose issue using this technique.

### EXAMPLE 2

### Dedifferentiation of Somatic Cells to Pluripotent Stem Cells In Vitro

Adult human keratinocytes are obtained form Clonetics (San Diego, Calif.) and grown in Keratinocyte Growth Media in 5-10% CO₂ at 37 degree C. according to the instructions “Keratinocyte System Instructions” BioWhittaker Catalogue number AA-1000. When the adult keratinocytes are about 40-80% confluent, 10-25 μM 5-aza-2′-deoxycytidine (Sigma, St Louis, Mo.) is added to the culture. After 4 days of incubation with 5-aza-2′-deoxycytidine, 100-250 ng/ml trichostatin (Sigma, St Louis, Mo.) is added to the culture. The culture is incubated for an additional day and sampled for telomerase activity.

Keratinocytes exposed to 10-25 μM 5-aza-2′-deoxycytidine for 5 days and 100-250 ng/ml trichostatin for 1 day and cells not exposed to these agents are assayed for the expression of telomerase (using a commercially available TRAP assay kit). These cells are assayed along side positive controls (HeLa and NH-12 cells) and negative controls (human foreskin fibroblasts and heated HN-12 cell extracts). Telomerase activity is measured by phosphoimaging telomeric products resolved on 12.5% polyacrylamide cells. Telometric ladders indicative of telomerase activity, consistent with the presence of stem cells is observed in the positive controls and in the keratinocytes exposed to 10-25 μM 5-aza-2′-deoxycytidine for 5 days and 100-250 ng/ml trichostatin for 1 day. These results confirm that keratinocytes exposed to 10-25 μM 5-aza-2′-deoxycytidine for 5 days and 100-250 ng/ml trichostatin for 1 day had increased telomerase activity consistent with a stem-cell like phenotype.

### EXAMPLE 3

### Growth of Stem Cells In Vitro

Stem cells were cultured in cell culture media composed of DMEM supplemented with 10% fetal bovine serum at 37 degree C. and 5% CO₂. Under these conditions cells can be passaged at least 5 times without differentiating, without losing their developmental phenotype.

### EXAMPLE 4

### Differentiation of Pluripotent Stem Cells into Specific Cell Type

A high density of stem cells (about 7×10⁶ cells/ml) is cultured for several weeks in media composed of: DMEN, supplemented with 1% FBS, 6.25 μM insulin, 6.25 μg/ml transferring, and 10 ng/ml transforming growth factor β1 (TGF-β-1), and 50 nM ascorbate 2-phosphate 1% ABAM.

After several weeks histological analysis of the tissue culture and paraffin sections is performed with H&E, alcain blue, toludene blue, and Goldner's trichrome staining at 2, 7, and 14 days. Samples are also tested for binding to antibodies raised against chondrotin-4-sulfate and keratin sulfate and type II collagen. A sample of the tissue culture is stained to obtain a qualitative estimate the amount of matrix present in the tissue culture. Control stem cells not exposed to the chondrogenic media show no evidence of differentiation into chondrogenic cells. Stem cells exposed to chondrogenic media show evidence of differentiation into chondrogenic cells forming cartilaginous spheroid nodules with a distinct border of perichondral cells as early as 48 hour after exposure to chondrogenic media.

### EXAMPLE 5

### Injecting Stem Cell/Collagen-rich Lattice Material Into Disc Space

Sufficient fatty tissue is removed from the patient via a lipo-suction procedure. The tissue is processed in such a way that stem cells and collagen-rich lattice, which are substantially free of adipocytes and red blood cells, can be separated from the rest of the tissue. The stem cells/collagen composition is injected directly into the nuclear disc space through an intact annulus using a small-diameter hypodermic needle. The stem cells/collagen composition is contained within the disc space following injection. Excess body fluid subsequently diffuses out of the disc space and leaves the stem cells-collagen lattice behind. Single injection is desirable; however, additional injections may be necessary to achieve appropriate level of physical augmentation and biological restoration.

### EXAMPLE 6

### Injecting Stem Cell/Collagen-Rich Lattice Material Into Disc Space

Sufficient fatty tissue is removed from the patient via a lipo-suction procedure. The tissue is processed in such a way that stem cells and collagen-rich lattice, which are substantially free of adipocytes and red blood cells, can be separated from the rest of the tissue. The collagen and stem cells are subsequently combined after separation during tissue processing. The stem cells/collagen composition may be further suspended in saline or any other appropriate medium. The suspension is injected directly into the nuclear disc space through an intact annulus using a hypodermic needle. The suspension is contained within the disc space following injection. The medium subsequently diffuses out of the disc space and leaves the stem cells-collagen lattice behind. Single injection is desirable; however additional injections may be necessary to achieve appropriate level of augmentation and restoration.

### EXAMPLE 7

### Injecting Stem Cell/Collagen-Rich Lattice Material Into Disc Space

Sufficient fatty tissue is removed from the patient via a lipo-suction procedure. The tissue is processed in such a way that stem cells and collagen-rich lattice, which are substantially free of adipocytes and red blood cells, can be separated from the rest of the tissue. The stem cells are expanded in-vitro and subsequently combined with the collagen. The stem cells/collagen composition may be further suspended in saline or any other appropriate medium. The suspension is contained within the disc space following injection. The medium subsequently diffuses out of the disc space and leaves the stem cells-collagen lattice behind. Single injection is desirable; however, additional injections may be necessary to achieve appropriate level of augmentation and restoration.

### EXAMPLE 8

### Adding a Radiocontrast Dye

Sufficient fatty tissue is removed from the patient via a lipo-suction procedure. The tissue is processed in such a way that stem cells and collagen-rich lattice, which are substantially free of adipocytes and red blood cells, can be separated from the rest of the tissue. A radiographic contrast dye is added to the stem cells/collagen composition before it is injected directly into the nuclear disc space through an intact annulus using a small-diameter hypodermic needle. The stem cells/collagen/dye composition is contained within the disc space following injection. Excess body fluid including the radiographic contrast dye subsequently diffuses out of the disc space and leaves the stem cells-collagen lattice behind. Single injection is desirable; however, additional injections may be necessary to achieve appropriate level of physical augmentation and biological restoration.

### EXAMPLE 9

### Adding an Analgesic Agent

Sufficient fatty tissue is removed from the patient via a lipo-suction procedure. The tissue is processed in such a way that stem cells and collagen-rich lattice, which are substantially free of adipocytes and red blood cells, can be separated from the rest of the tissue. An analgesic agent such as Lidocain is added to the stem cells/collagen composition before it is injected directly into the nuclear disc space through an intact annulus using a small-diameter hypodermic needle. The stem cells/collagen/analgesic composition is contained within the disc space following injection. Excess body fluid subsequently diffuses out of the disc space and leaves the stem cells-collagen lattice behind. Single injection is desirable; however, additional injections may be necessary to achieve appropriate level of physical augmentation and biological restoration.

EXAMPLE 10

### Adding a Growth Factor

Sufficient fatty tissue is removed from the patient via a lipo-suction procedure. The tissue is processed in such a way that stem cells and collagen-rich lattice, which are substantially free of adipocytes and red blood cells, can be separated from the rest of the tissue. One or more growth factors are added to the stem cells/collagen composition before it is injected directly into the nuclear disc space through an intact annulus using a small-diameter hypodermic needle. Preferred growth factors include those that may induce differentiation of stem cells into phenotypes that promote healing and/or regeneration of the disc. Examples of growth factors include transforming growth factor (TGF)-β, ascorbate-2-phosphate, bone morphogenetic proteins, fibroblast growth factors, platelet-derived growth factors, β-glycerophosphate, insulin, insulin-like growth factors, transferrin, hydrocortisone, etc. The stem cells/collagen/growth factor composition is contained within the disc space following injection. Excess body fluid subsequently diffuses out of the disc space and leaves the stem cells-collagen lattice behind. Single injection is desirable; however, additional injections may be necessary to achieve appropriate level of physical augmentation and biological restoration.

### EXAMPLE 11

### Adding Additional Collagen-Based Materials

Sufficient fatty tissue is removed from the patient via a lipo-suction procedure. The tissue is processed in such a way that stem cells and collagen-rich lattice, which are substantially free of adipocytes and red blood cells, can be separated from the rest of the tissue. One or more types of collagen are added to the stem cells/collagen composition before it is injected directly into the nuclear disc space through an intact annulus using a small-diameter hypodermic needle. Preferred collagen include those that are derived from collagen-rich or connective tissues such as intervertebral disc, fascia, ligament, tendon, demineralized bone matrix, etc. The stem cells/collagen composition is contained within the disc space following injection. Excess body fluid subsequently diffuses out of the disc space and leaves the stem cells-collagen lattice behind. Single injection is desirable; however, additional injections may be necessary to achieve appropriate level of physical augmentation and biological restoration.

### EXAMPLE 12

### Adding Polysaccharides

Sufficient fatty tissue is removed from the patient via a lipo-suction procedure. The tissue is processed in such a way that stem cells and collagen-rich lattice, which are substantially free of adipocytes and red blood cells, can be separated from the rest of the tissue. One or more types of polysaccharide are added to the stem cells/collagen composition before it is injected directly into the nuclear disc space through an intact annulus using a small-diameter hypodermic needle. Preferred polysaccharides include those that are derived from animals or vegetation such as hyaluronic acid, chitosan, chitin, cellulose, agar, etc. The stem cells/collagen/polysaccharide composition is contained within the disc space following injection. Excess body fluid subsequently diffuses out of the disc space and leaves the stem cells/collagen/polysaccharide matrix behind. Single injection is desirable; however, additional injections may be necessary to achieve appropriate level of physical augmentation and biological restoration.

### EXAMPLE 13

### Adding Additional Biomaterials

Sufficient fatty tissue is removed from the patient via a lipo-suction procedure. The tissue is processed in such a way that stem cells and collagen-rich lattice, which are substantially free of adipocytes and red blood cells, can be separated from the rest of the tissue. One or more biomaterials are added to the stem cells/collagen/composition before it is injected directly into the nuclear disc space through an intact annulus using a small-diameter hypodermic needle. Preferred biomaterials include albumin, fibrin, silk, elastin, keratin, and other synthetic hydrophilic polymers or hydrogels such as polyethylene oxide, polyethylene glycol, polyacrylamide, polyacrylic acid, polyvinyl alcohol, etc. The stem cells/collagen/biomaterial composition is contained within the disc space following injection. Excess body fluid subsequently diffuses out of the disc space and leaves the stem cells/collagen/biomaterial matrix behind. Single injection is desirable; however, additional injections may be necessary to achieve appropriate level of physical augmentation and biological restoration.

### EXAMPLE 14

### Adding a Multiple Additional Components

Sufficient fatty tissue is removed from the patient via a lipo-suction procedure. The tissue is processed in such a way that stem cells and collagen-rich lattice, which are substantially free of adipocytes and red blood cells, can be separated from the rest of the tissue. A radiographic contrast dye, an analgesic, a growth factor, a bulking agent and saline are added to the stem cells/collagen composition before it is injected directly into the nuclear disc space through an intact annulus using a small-diameter hypodermic needle. The bulking agent can be one or more of the collagens, polysaccharides, or biomaterials mentioned above. The final composition is contained within the disc space following injection. Excess body fluids including the radiographic contrast dye and saline subsequently diffuse out of the disc space and leaves the final matrix behind. Single injection is desirable; however, additional injections may be necessary to achieve appropriate level of physical augmentation and biological restoration.

### EXAMPLE 15

### Adding a Cross-Linking Agent

Sufficient fatty tissue is removed from the patient via a lipo-suction procedure. The tissue is processed in such a way that stem cells and collagen-rich lattice, which are substantially free of adipocytes and red blood cells, can be separated from the rest of the tissue. A cross-linking agent for collagen such as glutaraldehyde is added to the stem cells/collagen composition before it is injected directly into the nuclear disc space through an intact annulus using a small-diameter hypodermic needle. The collagen undergoes cross-linking in the disc space following injection. Excess body fluid subsequently diffuses out of the disc space and leaves the stem cells/cross-linked collagen matrix behind. Single injection is desirable; however, additional injections may be necessary to achieve appropriate level of physical augmentation and biological restoration.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected. Further examples of the inventive materials and methods are disclosed in applicant's co-pending U.S. patent application Ser. No. 10/245,955, which is incorporated herein by reference in its entirety.

1. A composition for treating an intervertebral disc, comprising stem cell material and a collagen-based material.

2. A composition according to claim 1 wherein said collagen-based material comprises a plurality of particles of a collagen-based material.

3. A composition according to claim 2 wherein said plurality of particles of a collagen-based material has a mean particle size of between 0.05 mm and 5.0 mm.

4. A composition according to claim 3 wherein said plurality of particles of a collagen-based material has a mean particle size of between 0.25 mm and 1.0 mm.

5. A composition according to claim 2 wherein said particles of a collagen-based material consists essentially of natural, collagen-rich tissue.

6. A composition according to claim 1 wherein said collagen-based material comprises a collagen-rich lattice.

7. A composition according to claim 6 wherein said stem cells are disposed in said collagen-rich lattice.

8. A composition according to claim 6 wherein said collagen-rich lattice material is harvested from the person into whom the composition is to be surgically added.

9. A composition according to claim 1 wherein said treating composition additionally includes a second agent selected from the group consisting of analgesics, antibiotics, radiocontrast materials, growth factors, crosslinking agents, polysaccharides, hormones, proteoglycans, and agents effective for treating degenerative disc disease.

10. A composition according to claim 9 wherein said second agent comprises one or more growth factors selected from the group consisting of transforming growth factor (TGF)-β, ascorbate-2-phosphate, bone morphogenetic proteins, fibroblast growth factors, platelet-derived growth factors, β-glycerophosphate, insulin, insulin-like growth factors, transferrin, and hydrocortisone.

11. A composition according to claim 9 wherein said second agent comprises one or more hormones selected from the group consisting of cytokines and morphogens.

12. A composition according to claim 1 wherein said stem cell material consists essentially of autologous stem cell material.

13. A composition according to claim 1 wherein said stem cell material consists essentially of human allogenic stem cell material.

14. A composition according to claim 1 wherein said stem cell material consists essentially of xenogenic stem cell material.

15. A composition according to claim 1 wherein said stem cell material consists essentially of stem cells that have never begun to differentiate.

16. A composition according to claim 1 wherein said stem cell material consists essentially of stem cells that have begun to differentiate but have been returned to an undifferentiated or substantially undifferentiated state.

17. A composition according to claim 1 wherein said stem cell material consists essentially of stem cells that have begun to differentiate, but have been returned to a partially-differentiated state.

18. A composition according to claim 1 wherein said stem cell material comprises stem cells that express or have been induced to express at least one characteristic of human intervertebral disc cells.

19. A composition according to claim 18 wherein said stem cell material has been induced to express at least one characteristic of human intervertebral disc cells by contacting the stem cell material with a member selected from the group consisting of transforming growth factor (TGF)-β, ascorbate-2-phosphate, bone morphogenetic proteins, fibroblast growth factors, platelet-derived growth factors, β-glycerophosphate, insulin, insulin-like growth factors, transferrin, and hydrocortisone.

20. A composition according to claim 18 wherein said stem cell material has been induced to express at least one characteristic of human intervertebral disc cells by contacting the stem cell material with mature cells of a desired phenotype.

21. A composition according to claim 1 wherein said stem cell material comprises stem cells that express or have been induced to express at least one characteristic of fibroblast cells.

22. A composition according to claim 1 wherein said stem cell material comprises stem cells that express or have been induced to express at least one characteristic of chondrocyte cells.

23. A composition according to claim 1 wherein said stem cell material comprises stem cells that express or have been induced to express at least one characteristic of notochordal cells.

24. A composition according to claim 1 wherein said stem cell material has been separated from non-stem cell material before being added to an intervertebral disc.

25. A composition according to claim 24 wherein said stem cell material is substantially free of non-stem cell material.

26. A composition according to claim 1 wherein said composition further includes a material effective for inducing stem cells to differentiate into intervertebral disc cells.

27. A composition according to claim 26 wherein said material effective for inducing stem cells to differentiate into intervertebral disc cells comprises a member selected from the group consisting of transforming growth factor (TGF)-β, ascorbate-2-phosphate, bone morphogenetic proteins, fibroblast growth factors, platelet-derived growth factors, β-glycerophosphate, insulin, insulin-like growth factors, transferrin, and hydrocortisone.

28. A composition according to claim 26 wherein said material effective for inducing stem cells to differentiate into intervertebral disc cells comprises mature cells of a desired phenotype.

29. A method of treating an intervertebral disc, said method comprising surgically adding to an intervertebral disc a treating composition comprising stem cell material and a collagen-based material.

30. An intervertebral disc that has been treated to repair damage done by degenerative disc disease, said disc comprising a disc annulus having contained therein a treating composition comprising stem cell material and a collagen-based material.
